(19) United States
(12) Patent Application Publication
(10) Pub. No.: US 2005/0118128 A1
(43) Pub. Date: Jun. 2, 2005

(54) Disinfection efficacy of lens care regimen

(76) Inventors: Roya Nicole Borazjani, Fairport, NY (US); Richard Smerbeck, Pittsford, NY (US); Daniel M. Ammon JR., Rochester, NY (US); Joseph C. Salamone, Fairport, NY (US)

Correspondence Address: RITA D. VACCA;BAUSCH & LOMB INCORPORATED, ONE BAUSCH & LOMB PLACE, ROCHESTER, NY, 14604-2701, US

(21) Appl. No.: 10/724,797

(22) Filed: Dec. 1, 2003

(51) Int. Cl. A61K 7/06; A61K 7/11; A61K 31/13

(52) U.S. Cl. 424/70.11; 514/667

(57) ABSTRACT

Novel compositions and methods for using lens care solutions made from the compositions to effectively clean and disinfect contact lenses. The methods for effectively cleaning and disinfecting contact lenses require no lens rubbing a step and require no lens rinsing step.

### FIELD OF THE INVENTION

The present invention is directed toward novel compositions and methods for disinfecting contact lenses. More specifically, the subject invention is directed toward compositions and methods for disinfecting contact lenses that require no lens rubbing step and require no lens rinsing step.

### BACKGROUND OF THE INVENTION

Generally, a care regimen for contact lenses involves various functions, such as regularly cleaning the lens with a contact lens solution containing a surface-active agent as a primary cleaning agent. Rinsing of the contact lens is generally recommended following cleaning to remove loosened debris. Additionally, the regimen may include treatment to disinfect the lens, treatment to render the lens surface more wettable prior to insertion in the eye and/or treatment to condition, e.g., lubricate or cushion, the lens surface so that the lens is more comfortable in the eye. As a further example, a contact lens wearer may need to rewet the lens during wear by administering directly in the eye a solution commonly referred to as rewetting drops.

Separate solutions may be provided for the individual segments of the care regimen. For convenience purposes, multipurpose contact lens solutions have gained popularity, i.e., solutions that can be used for several segments of the care regimen.

Multipurpose contact lens solutions that effectively clean a contact lens and can also be used to treat the lens immediately prior to insertion of the lens in the eye or while the lens is worn in the eye, represent the more difficult multipurpose solutions to develop. Such solutions are difficult to develop since the solutions come into direct contact with eye tissue and tear film. Conventional surface active agents having good cleaning activity for contact lens deposits, as well as various other components such as antimicrobial agents included as a preservative or disinfectant, tend to be irritating to the eye. Additionally, the surface-active agents must not inhibit the wetting or conditioning function of the solution.

U.S. Pat. No. 5,604,189 discloses multi-purpose compositions for cleaning and wetting contact lenses that include a poly(ethylene oxide)-containing material having a hydrophilic/lipophilic balance (HLB) of at least about 18, and a surface active agent having cleaning activity for contact lens deposits. The compositions provide effective cleaning activity, and are also effective at wetting surfaces of the lens. Additionally, the compositions achieve the desired cleaning while being relatively nonirritating to the eye. According to preferred embodiments, the compositions are sufficiently nonirritating that contact lenses treated with the compositions can be inserted directly in the eye, i.e., without the need to rinse the compositions from the lens, or the compositions can be administered directly in the eye for use as rewetting solutions. Compositions of the type disclosed in Table 16 of the '189 patent and marketed under the trade name Simplicity™ (Polymer Technology, Rochester, N.Y.) have shown commercial success as a multi-purpose solution for cleaning, conditioning, wetting and disinfecting rigid gas permeable (RGP) contact lenses.

One type of product that would require more efficacious disinfection is a multi-purpose solution that would not require digital rubbing of the contact lens with the solution as part of its regimen of use. With conventional contact lens cleaners and disinfectants, including multi-purpose solutions, lens wearers typically need to digitally or manually rub the contact lenses, typically between a finger and palm or between fingers, during treatment of the contact lenses. The necessity for the daily “rubbing” of contact lenses adds to the time and effort involved in the daily care of contact lenses. Many contact-lens wearers dislike having to perform such a regimen or consider it to be inconvenient. Some wearers may be negligent in the proper “rubbing” regimen, which may result in contact lens discomfort and other problems. Sometimes rubbing, if performed too vigorously, which is particularly apt to occur with beginning lens wearers, may damage the lenses. This can be especially problematic when a replacement lens is not immediately available.

Contact lens solutions that qualify as a “Chemical Disinfecting Solution” do not require rubbing to meet biocical performance criteria for destroying representative bacteria and fungi, as set by the U.S. Food and Drug Administration (FDA) under the Premarket Notification (510 k) Guidance Document for Contact Lens Care Products, May 1, 1997. In contrast, a contact lens solution, referred to as a “Chemical Disinfecting System,” not qualifying as a Chemical Disinfecting Solution, requires a rubbing regimen to pass biocidal performance criteria. Traditionally, multi-purpose solutions used for disinfecting and wetting or for disinfecting, cleaning and wetting, have qualified as a Chemical Disinfecting System, but not as a Chemical Disinfecting Solution.

Traditional contact lens multi-purpose solutions may depend on a rubbing regimen, not only for efficacious disinfection, but also for efficacious cleaning. Efficacious cleaning also requires a rinsing step to remove loosened debris. Thus, in order to develop a contact lens care solution that would not require rubbing and would not require rinsing, both improved or stronger cleaning and disinfection may be needed, while at the same time maintaining the solution sufficiently gentle for in-the-eye use.

Thus, it would be desirable to obtain a multi-purpose contact lens solution that would provide increased disinfecting efficacy. Further, it would be desirable to obtain improved cleaning efficacy while maintaining or increasing the biocidal efficacy of the product without adversely affecting the comfort or safety in terms of the level of toxicity to eye tissue. While still more challenging to develop, it would also be desirable to develop a multi-purpose solution that exhibits both efficacious cleaning and disinfection of a contact lens, without requiring a rubbing regimen and without requiring a rinsing regimen.

### SUMMARY OF THE INVENTION

The present invention is directed to contact lens care compositions and methods of using the compositions in a “no rub and no rinse” regimen for cleaning and disinfecting contact lenses. Such lens care compositions and methods of the present invention allow for a more convenient lens care regimen that eliminates the need for digitally or manually rubbing the contact lenses, typically between a finger and palm or between fingers, during treatment of the contact lenses. Thus, through elimination of the necessity for daily “rubbing” of contact lenses, the time and effort involved in the daily care of contact lenses is reduced. Likewise, compositions and methods of the present invention eliminate the need for a rinsing step to remove loosened debris, while remaining gentle enough for in-the-eye use.

Methods of the present invention require but two of the following four regimen steps for effective cleaning and disinfection of contact lenses:
using an increased total volume of lens care composition or solution for lens soaking;
adding a lens care composition or solution to a lens case after placement of a lens therein;
shaking, revolving or otherwise agitating a lens case containing a lens and a lens care composition or solution; and
soaking a lens in a lens case with a lens care composition or solution for an extended period of time.
   Through studies, it was found that a combination of two or more of the above-described regimen steps were needed to comply with the U.S. FDA requirements for a no rub and no rinse multi-purpose disinfecting solution for contact lenses. It is important to note that the U.S. FDA requirements for a no rub and no rinse multi-purpose disinfecting solution for contact lenses is considerably more stringent than the regulatory requirements for no rub and no rinse in other countries.

### DETAILED DESCRIPTION OF THE INVENTION

The present invention is directed to contact lens care compositions useful in a “no rub and no rinse” regimen for cleaning and disinfecting contact lenses. Contact lens care compositions or solutions require disinfection compliance with the FDA under the Premarket Notification (510 k) Guidance Document for Contact Lens Care Products, May 1, 1997 and ISO 14729, International Standardized Document for Ophthalmic Optics. These guidelines utilize two steps, namely a stand-alone disinfection part and a regimen test procedure part. The stand-alone procedure measures the extent of viability loss of representative microorganisms at established time intervals to determine the extent of viability loss. The regimen test procedure is applicable to multi-functional disinfection solutions, which may include cleaning, rinsing and soaking, and is accomplished based on the manufacturing recommended manner.

The test organisms recommended by the FDA 510(k) Guidance Document and ISO 14729 include three bacteria, i.e., Pseudomonas aeruginosa ATCC 9027, Stapylococcus aureus ATCC 6538 and Serratia marcescens ATCC 13880, and two fungi, i.e., Candida albicans ATCC 10231 and Fusarium solani ATCC 36031. The performance requirement for regimen requires recovery of less than or equal to 10 colony-forming units (CFU) from each lens and filter combination for each test organism.

To determine whether a “no rub and no rinse” regimen was feasible for disinfecting contact lenses, group I and group IV lenses were studied in conjunction with several permitted variables in the FDA regimen test procedure. While meeting the requirements of the FDA 510(k) Guidance Document and ISO 14729, certain steps of the regimen test procedure are not specified and are open to suitable alternatives within the guidance document's limitations. In lieu of this, the following regimen test procedure modifications were studied.
1) The total volume of lens disinfecting solution used for the four hour soaking time was increased from 3 ml to 5 ml or greater.
2) The lens care solution was added to a lens case after placing a lens in the case as opposed to current systems where a lens is placed in a lens case previously filled with solution.
3) The lens case containing the lens was shaken, revolved or otherwise agitated for 5 to 10 seconds.
4) The soaking time for the lens in the lens case was increased from 4 hours to 6 hours.
   In the study, it was found that a combination of two or more of the modifications described above were needed to comply with the FDA requirements for a no rub and no rinse multi-purpose disinfecting solution for contact lenses with the five recommended test organisms identified above.

In the regimen test, organic soil or artificial tear model is added to the lenses to mimic deposits that may be present in actual patient use situation. Inclusion of organic load allows for an evaluation of the cleaning step to remove debris and associated microorganisms, as well as the interaction of any remaining organic material with the soaking solution. According to ISO International Standards for Ophthalmic Optics (ISO 14729), since the addition of organic soil has not been standardized for use in the regimen method at this time, an artificial tear or organic soil is not required during the evaluation of contact lens care products. The United States Food and Drug Administration (FDA) on the other hand, recommends the use of organic soil for product registration in the United States.

Novel compositions of the present invention are gentle enough to be ophthalmically compatible for in-the-eye use without a rinsing step while providing effective disinfection in a no rub-no rinse regimen. In formulating such compositions for no rub/no rinse lens care solutions, key ingredients required to achieve both disinfecting effectiveness and gentleness are one or more hydroxyalkylamines, one or more polyols, one or more polymer surfactants and one or more disinfecting agents. The one or more hydroxyalkylamines suitable for use in compositions of the present invention have C₁₋₆ alkyl groups and more preferably C₁₋₃ alkyl groups. Such suitable one or more hydroxyalkylamines include for example primary, secondary or tertiary amines but most preferably tertiary amines such as for example but not limited to triethanolamine. The preferred total concentration of one or more hydroxyalkylamines present in the subject compositions is approximately 0.1 to 5.0 weight percent and more preferably approximately 0.5 to 3.0 weight percent.

Compositions of the present invention likewise include one or more C₁₋₃₆ polyols such as for example but not limited to glycerin or ethylene glycol but most preferably glycerin. The lowest possible volume of one or more polyols capable of achieving the desired gentleness, is used in the subject compositions. Typically the lowest possible volume suitable to achieve the desired gentleness, i.e., to obtain a solution osmolarity within the range of approximately 220 to 380 mOsm/kg, is approximately 0.5 weight percent or greater. It is important to note that the lowest possible volume of one or more polyols is used in the subject compositions since increasing volumes of polyols can decrease the effectiveness of disinfecting agents within the composition.

Compositions of the present invention also include one or more polymeric surfactants having a hydrophilic/lipophilic balance (HLB) of 20 or above. Suitable polymeric surfactants include for example but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO, such as for example poloxamers and poloxamines, are commercially available under the trade names Pluronic™ and Tetronic™ (BASF Wyandotte Corp., Wyandotte, Mich.). Preferred polymeric surfactants include but are not limited to Pluronic F38 and Tetronic 908. The preferred total concentration of one or more polymeric surfactants in the subject compositions is approximately 0.5 to 5.0 weight percent.

Compositions of the present invention also include one or more disinfecting agents to achieve effective disinfection in a no rub-no rinse regimen. Suitable disinfecting agents include for example but are not limited to 1,1′-hexamethylene-bis[5-(p-chlorophenyl)biguanide] (Chlorhexidine), water soluble salts of Chlorhexidine, 1,1′-hexamethylene-bis[5-(2-ethylhexyl)biguanide] (Alexidine), water soluble salts of Alexidine, poly(hexamethylene biguanide) (PHMB), water soluble salts of PHMB, propyl-4-hydroxybenzoate (PHB), quaternary ammonium esters and the like. Biguanides are described in U.S. Pat. Nos. 5,990,174; 4,758,595 and 3,428,576 each incorporated herein in its entirety by reference. The preferred biguanide due to its ready commercial availability is poly(aminopropyl biguanide) (PAPB), also commonly referred to as poly(hexamethylene biguanide) (PHMB). Preferred disinfecting agents include PHMB and Alexidine. If disinfecting agents such as PHMB and Alexidine are used in combination, the total concentration of disinfecting agent is preferably within the range of approximately 3 ppm to 6 ppm. More preferably a combination of disinfecting agents includes approximately 0.1 to 1.0 ppm PHMB and approximately 3.0 to 6.0 ppm Alexidine. Most preferably a combination of disinfecting agents includes approximately 0.5 ppm PHMB and 3.0 ppm Alexidine to approximately 0.7 ppm PHMB and 4.0 ppm Alexidine. If PHMB should be used alone, the preferred concentration is approximately 0.5 to 1.1 ppm. Should Alexidine be used alone, the preferred concentration is approximately 4.0 to 6.0 ppm.

Compositions of the present invention have a pH of about 6.0 to 8.0, and more preferably a pH of about 6.5 to 7.8. To adjust the final pH, one or more suitable buffers may be added to the subject solutions such as but not limited to ethanolamine, diethanolamine, triethanolamine, tromethamine, borate, citrate, phosphate, bicarbonate, and various mixed buffers or buffer systems. Generally, buffers will be used in amounts ranging from about 0.05 to 2.5 percent by weight, and preferably from 0.1 to 1.5 percent by weight.

Compositions or ophthalmic solutions of the present invention may also include one or more tonicity adjusting agents, optionally in the form of a buffering agent, for providing an isotonic or close to isotonic solution such that the osmolarity is about 200 to 400 mOsm/kg, but preferably about 220 to 380 mOsm/kg and most preferably about 250 to 350 mOsm/kg. Examples of suitable tonicity adjusting agents include but are not limited to sodium chloride, potassium chloride, dextrose, mannose, glycerin, propylene glycol, calcium chloride and magnesium chloride. These agents are typically used individually in amounts ranging from about 0.01 to 2.5 weight percent and preferably from about 0.1 to about 1.5 weight percent.

It may also be desirable to optionally include in the subject compositions or solutions one or more water soluble viscosity builders such as for example but not limited to hydroxypropylmethyl cellulose, hydroxyethyl cellulose, poly(N-vinylpyrrolidone) (PVP) and poly(vinyl alcohol). Because of their demulcent effect, viscosity builders have a tendency to further enhance the lens wearer's comfort by means of a film on the lens surface cushioning impact against the eye.

Compositions of the present invention may likewise include one or more sequestering agents to bind metal ions, which in the case of ophthalmic solutions, might otherwise react with protein deposits and collect on contact lenses. Suitable sequestering agents include for example but are not limited to ethylenediaminetetraacetic acid (EDTA) and its salts. Sequestering agents are preferably used in amounts ranging from about 0.01 to about 0.2 weight percent.

Compositions of the present invention may also include one or more polysaccharides. One or more polysaccharides are present in the subject compositions in a total amount of from approximately 0.01 to approximately 3.0 percent by weight based on the total weight of the composition, but more preferably from about 0.02 to about 2.0 percent by weight. Suitable polysaccharides for use in compositions of the present invention include for example but are not limited to trehalose, variations of polyquaternium-10 such as for example but not limited to Polymer JR 30M™ (Dow Chemical Company, Midland, Mich.), and variations of polyquaternium-16 and polyquaternium-44 such as for example but not limited to Luviqua™ (BASF Wyandotte Corp).

Specific compositions of the present invention and no rub and no rinse studies and study results are described in still greater detail in the examples provided below. However, it is to be understood that the following examples are for illustrative purposes only and do not purport to be wholly definitive as to conditions and scope of the present invention.

### EXAMPLE 1

### Preparation of Test Sample Solutions

Sample solutions for testing were prepared in accordance with the formulations set forth below in Table 1.

TABLE 1
Test Sample Solutions

| Ingredients (w/w %) | Sample 1 | Sample 2 | Sample 3 |
|---|---|---|---|
| Triethanolamine HCl 99.5% | 0.937 | 0.937 | 0.937 |
| Triethanolamine 98% | 0.149 | 0.149 | 0.149 |
| Pluronic F38 | 1 | 1 | 1 |
| Tetronic 908 | 1 | 1 | 1 |
| PVP | 1 | 1 | 1 |
| EDTA | 0.025 | 0.025 | 0.025 |
| Glycerin | 0.722 | 0.722 | 0.722 |
| NaCl | 0.05 | 0.05 | 0 |
| Polymer JR 30 M | 0.02 | 0.02 | 0.02 |
| PHMB | 0.7 ppm | 0 | 0 |
| PHB | 50 ppm | 50 ppm | 50 ppm |
| Trehalose | 0 | 0 | 0.2 |
| Alexidine | 4 ppm | 4.5 ppm | 4.5 ppm |
| pH | 7.14 | 7.10 | 7.00 |
| Osmolarity | 226 | 230 | 220 |
| Ingredients (w/w %) | Sample 4 | Sample 5 | Sample 6 |
| Triethanolamine HCl 99.5% | 0.937 | 0.937 | 0.937 |
| Triethanolamine 98% | 0.149 | 0.149 | 0.149 |
| Pluronic F38 | 1 | 1 | 1 |
| Tetronic 908 | 1 | 1 | 1 |
| PVP | 1 | 0 | 1 |
| EDTA | 0.025 | 0.025 | 0.025 |
| Glycerin | 0.722 | 0.722 | 0.722 |
| NaCl | 0 | 0.05 | 0.05 |
| Polymer JR 30 M | 0.02 | 0.02 | 0.02 |
| PHMB | 0.7 ppm | 0 | 0 |
| PHB | 0 | 0 | 0 |
| Trehalose | 0.2 | 0 | 0 |
| Alexidine | 4 ppm | 4.5 ppm | 4.5 ppm |
| pH | 7.11 | 7.13 | 7.09 |
| Osmolarity | 213 | 224 | 234 |

| Ingredients (w/w %) | Sample 7 | Sample 8 |
|---|---|---|
| Triethanolamine HCl 99.5% | 0.937 | 0.937 |
| Triethanolamine 98% | 0.149 | 0.149 |
| Pluronic F38 | 1 | 1 |
| Tetronic 908 | 1 | 1 |
| PVP | 1 | 1 |
| EDTA | 0.025 | 0.025 |
| Glycerin | 0.722 | 0.722 |
| NaCl | 0.05 | 0.05 |
| Polymer JR 30 M | 0.02 | 0.02 |
| PHMB | 0 | 0 |
| PHB | 0 | 0 |
| Luviquat | 0.05 | 0.1 |
| Trehalose | 0.2 | 0.2 |
| Alexidine | 4.5 ppm | 4.5 ppm |
| pH | 7.16 | 7.16 |
| Osmolarity | 218 | 218 |

### EXAMPLE 2

### Stand-Alone Biocidal Testing and “No Rub-No Rinse” Regimen with A Shaking Step Testing with Five of FDA/ISO Challenge Microorganisms on Two Different Group IV Lenses

A four-hour no rub and no rinse (NR/NR) regimen using 10 ml of sample solution with a 10 second shaking step (ss) was conducted on Focus™ Monthly (CIBA Vision, Basel, Switzerland) Group IV lenses (Gr IV-A), and Surevue™ (Johnson & Johnson, New Brunswick, N.J.) Group IV lenses (Gr IV-B) and tested using Candida albicans ATCC 10231. The test results for the regimens are set forth below in Table 2. A Stand-Alone Biocidal study using 10 percent organic soil was also conducted whereby the samples were tested against Pseudomas aeruginosa ATCC 9027, Staphylococcus aureus ATCC 6538, Serratia marcescens ATCC13880, Candida albicans ATCC 10231 and Fusarium solani ATCC 36031. The results of the Stand-Alone Biocidal study are also set forth below in Table 2.

TABLE 2
Efficacy of Various Test Solutions in No Rub/No Rinse (NR/NR)
Regimen And Stand-Alone Biocidal Testing

| TEST | Sample 1 | Sample 2 | Sample 3 |
|---|---|---|---|
| NR/NR Regimen | 1, 1, 1 | 2, 1, 0 | 0, 4, 4 |
| 4 Hr soak/10 ml/10 ss (Gr IV-A) | | | |
| Candida albicans (CFU) | | | |
| NR/NR Regimen | 3, 10, 8 | 5, 0, 2 | 1, 2, 3 |
| 4 Hr soak/10 ml/10 ss (Gr IV-B) | | | |
| Candida albicans (CFU) | | | |

<10 CFU = test passage
>10 CFU = test failure
CFU = colony forming units

| Stand-Alone Biocidal | | | |
|---|---|---|---|
| (10% organic soil) | Log Reduction | | |
| Pseudomas aeruginosa | | | |
| 1 Hour Soaking Time | >4.7 | >4.8 | >4.8 |
| 4 Hour Soaking Time | >4.7 | >4.8 | >4.8 |
| Staphylococcus aureus | | | |
| 1 Hour Soaking Time | >4.6 | >4.7 | >4.7 |
| 4 Hour Soaking Time | >4.6 | >4.7 | >4.7 |
| Serratia marcescens | | | |
| 1 Hour Soaking Time | >4.9 | 3.9 | >4.6 |
| 4 Hour Soaking Time | >4.9 | >4.6 | >4.6 |
| Candida albicans | | | |
| 1 Hour Soaking Time | 1.9 | >4.8 | >4.8 |
| 4 Hour Soaking Time | 3.3 | >4.8 | >4.8 |
| Fusarium solani | | | |
| 1 Hour Soaking Time | >4.3 | 3.1 | 2.4 |
| 4 Hour Soaking Time | >4.3 | >4.4 | >4.4 |

Log Reduction: >= 100 percent kill

### EXAMPLE 3

### Stand-Alone Biocidal Testing and “No Rub-No Rinse” Regimen with a Shaking Step Testing with Five of FDA/ISO Challenge Microorganisms

A four-hour no rub and no rinse (NR/NR) regimen using 10 ml of sample solution or 8 ml of sample solution, and a 10 second shaking step (ss) was conducted on Focus™ Monthly (CIBA Vision, Basel, Switzerland) Group IV lenses (Gr IV-A). The lenses were then tested using Candida albicans ATCC 10231. The test results for the regimens are set forth below in Table 3. A Stand-Alone Biocidal study using 10 percent organic soil was also conducted whereby the samples were tested against Pseudomas aeruginosa ATCC 9027, Staphylococcus aureus ATCC 6538, Serratia marcescens ATCC13880, Candida albicans ATCC 10231 and Fusarium solani ATCC 36031. The results of the Stand-Alone Biocidal study are also set forth below in Table 3.

TABLE 3
Efficacy of Various Test Solutions in No Rub/No Rinse Regimen
and Stand-Alone Biocidal Testing

| TEST | Sample 2 | Sample 4 |
|---|---|---|
| NR/NR Regimen | 2, 1, 12 | 0, 3, 3 |
| 4 Hr soak/10 ml/10 ss (Gr IV-A) | | |
| Candida albicans (CFU) | | |
| 4 Hr soak/8 ml/10 ss (Gr IV-A) | 0, 1, 3 | 2, 1, 0 |
| Candida albicans (CFU) | | |

<10 CFU = test passage
>10 CFU = test failure
CFU = colony forming units

| Stand-Alone Biocidal | | |
|---|---|---|
| (10% organic soil) | Log Reduction | |
| Pseudomas aeruginosa | | |
| 1 Hour Soaking Time | >4.8 | >4.9 |
| 4 Hour Soaking Time | >4.8 | >4.9 |
| Staphylococcus aureus | | |
| 1 Hour Soaking Time | >4.7 | >4.0 |
| 4 Hour Soaking Time | >4.7 | >4.2 |
| Serratia marcescens | | |
| 1 Hour Soaking Time | 3.9 | >3.8 |
| 4 Hour Soaking Time | >4.6 | >3.8 |
| Candida albicans | | |
| 1 Hour Soaking Time | >4.8 | 4.1 |
| 4 Hour Soaking Time | >4.8 | >4.8 |
| Fusarium solani | | |
| 1 Hour Soaking Time | >4.4 | 2.7 |
| 4 Hour Soaking Time | >4.4 | >4.4 |

Log Reduction: >= 100 percent kill

### EXAMPLE 4

### Stand-Alone Biocidal Testing and “No Rub-No Rinse” Regimen with a Shaking Step Testing with Five of FDA/ISO Challenge Microorganisms on Two Different Group IV Lenses

A four-hour no rub and no rinse (NR/NR) regimen using 10 ml of sample solution with a 10 second shaking step (ss) was conducted on Focus™ Monthly (CIBA Vision, Basel, Switzerland) Group IV lenses (Gr IV-A), and Surevue™ (Johnson & Johnson, New Brunswick, N.J.) Group IV lenses (Gr IV-B) and tested using Candida albicans ATCC 10231. The test results for the regimens are set forth below in Table 4. A Stand-Alone Biocidal study using 10 percent, 50 percent, 100 percent and no organic soil was also conducted whereby the samples were tested against Pseudomas aeruginosa ATCC 9027, Staphylococcus aureus ATCC 6538, Serratia marcescens ATCC13880, Candida albicans ATCC 10231 and Fusarium solani ATCC 36031. The results of the Stand-Alone Biocidal study are also set forth below in Table 4.

TABLE 4
Efficacy of Various Test Solutions in No Rub/No Rinse Regimen and
Stand-Alone Biocidal Testing Using Various Concentrations of Organic
Soil

| TEST | Sample 3 | Sample 5 | Sample 6 |
|---|---|---|---|
| NR/NR Regimen | 2, 0, 1 | 1, 0, 1 | 0, 0, 3 |
| 4 Hr soak/10 ml/10 ss (Gr IV-A) | | | |
| Candida albicans (CFU) | | | |
| 4 Hr soak/10 ml/10 ss (Gr IV-B) | 8, 4, 6 | 9, 4, 5 | 11, 5, 6 |
| Candida albicans (CFU) | | | |

<10 CFU = test passage
>10 CFU = test failure
CFU = colony forming units

| Stand-Alone Biocidal | | | |
|---|---|---|---|
| (10% organic soil) | Log Reduction | | |
| Pseudomas aeruginosa | | | |
| 1 Hour Soaking Time | >4.8 | 4.9 | >4.9 |
| 4 Hour Soaking Time | >4.8 | >4.9 | >4.9 |
| Staphylococcus aureus | | | |
| 1 Hour Soaking Time | >4.7 | >4.8 | 4.2 |
| 4 Hour Soaking Time | >4.7 | >4.8 | 4.2 |
| Serratia marcescens | | | |
| 1 Hour Soaking Time | >4.6 | 3.6 | >3.8 |
| 4 Hour Soaking Time | >4.6 | >4.8 | >3.8 |
| Candida albicans | | | |
| 1 Hour Soaking Time | >4.8 | 2.6 | 4.9 |
| 4 Hour Soaking Time | >4.8 | >4.8 | >4.9 |
| Fusarium solani | | | |
| 1 Hour Soaking Time | 2.4 | >4.4 | >4.4 |
| 4 Hour Soaking Time | >4.4 | >4.4 | >4.4 |
| Stand-Alone Biocidal | | | |
| (50% organic soil) | Log Reduction | | |
| Pseudomas aeruginosa | | | |
| 1 Hour Soaking Time | 3.3 | 3.8 | 3.4 |
| 4 Hour Soaking Time | 3.7 | 5.0 | 5.0 |
| Staphylococcus aureus | | | |
| 1 Hour Soaking Time | >4.9 | >4.9 | >4.9 |
| 4 Hour Soaking Time | >4.9 | >4.9 | 4.7 |
| Serratia marcescens | | | |
| 1 Hour Soaking Time | >4.7 | 4.2 | >4.7 |
| 4 Hour Soaking Time | >4.7 | >4.7 | >4.7 |
| Candida albicans | | | |
| 1 Hour Soaking Time | 3.0 | 2.2 | 3.1 |
| 4 Hour Soaking Time | >5.0 | 4.3 | 4.3 |
| Fusarium solani | | | |
| 1 Hour Soaking Time | >4.9 | >4.9 | >4.9 |
| 4 Hour Soaking Time | >4.9 | >4.9 | >4.9 |
| Stand-Alone Biocidal | | | |
| (100% organic soil) | Log Reduction | | |
| Pseudomas aeruginosa | | | |
| 1 Hour Soaking Time | 2.2 | 2.3 | 2.3 |
| 4 Hour Soaking Time | 2.9 | 3.2 | 3.2 |
| Staphylococcus aureus | | | |
| 1 Hour Soaking Time | >4.8 | >4.8 | >4.8 |
| 4 Hour Soaking Time | >4.8 | >4.8 | >4.8 |
| Serratia marcescens | | | |
| 1 Hour Soaking Time | >4.3 | 2.6 | 2.4 |
| 4 Hour Soaking Time | >4.3 | >4.3 | >4.3 |
| Candida albicans | | | |
| 1 Hour Soaking Time | 3.1 | 2.6 | 2.8 |
| 4 Hour Soaking Time | 4.5 | 4.8 | 4.5 |
| Fusarium solani | | | |
| 1 Hour Soaking Time | 2.3 | 3.1 | 4.5 |
| 4 Hour Soaking Time | >4.9 | >4.9 | >4.9 |
| Stand-Alone Biocidal | | | |
| (No organic soil for Only 3 minutes) | Log Reduction | | |
| Pseudomas aeruginosa | | | |
| 45 Second Soaking Time | 3.1 | 3.2 | 3.3 |
| 90 Second Soaking Time | 3.2 | 3.6 | 4.0 |
| 135 Second Soaking Time | 3.5 | 3.5 | 4.0 |
| 180 Second Soaking Time | 2.9 | 3.1 | 3.2 |
| Staphylococcus aureus | | | |
| 45 Second Soaking Time | 4.7 | >4.9 | >4.9 |
| 90 Second Soaking Time | 4.6 | 4.9 | >4.9 |
| 135 Second Soaking Time | 4.9 | 3.2 | >4.9 |
| 180 Second Soaking Time | 4.9 | 4.9 | >4.9 |
| Serratia marcescens | | | |
| 45 Second Soaking Time | 1.0 | 1.6 | 1.5 |
| 90 Second Soaking Time | 1.1 | 2.1 | 1.8 |
| 135 Second Soaking Time | 1.9 | 2.2 | 2.3 |
| 180 Second Soaking Time | >4.2 | 3.7 | >4.2 |
| Candida albicans | | | |
| 45 Second Soaking Time | 1.8 | 2.1 | 2.3 |
| 90 Second Soaking Time | 2.1 | 2.8 | 2.8 |
| 135 Second Soaking Time | 2.6 | 3.0 | 3.2 |
| 180 Second Soaking Time | 2.7 | 3.3 | 3.2 |
| Fusarium solani | | | |
| 45 Second Soaking Time | 1.5 | 1.8 | 1.4 |
| 90 Second Soaking Time | 2.2 | 1.7 | 1.1 |
| 135 Second Soaking Time | 2.8 | 1.3 | 2.4 |
| 180 Second Soaking Time | 2.8 | 1.8 | 1.7 |

Log Reduction: > = 100 percent kill

### EXAMPLE 5

### “No Rub-No Rinse” Regimen with and without a Shaking Step Testing with Four of FDA/ISO Challenge Microorganisms on Two Different Group IV Lenses Using Test Solution 1

A four-hour no rub and no rinse (NR/NR) regimen using 10 ml of test sample solution 1 with and without a 10 second shaking step (ss) was conducted on Focus™ Monthly (CIBA Vision, Basel, Switzerland) Group IV lenses (Lens Sample A), and Surevue™ (Johnson & Johnson, New Brunswick, N.J.) Group IV lenses (Lens Sample B) and tested using Pseudomas aeruginosa ATCC 9027, Staphylococcus aureus ATCC 6538, Serratia marcescens ATCC13880, and Fusarium solani ATCC 36031. The test results for the regimens are set forth below in Table 5.

TABLE 5
Results of No Rub-No Rinse Regimen With And Without Shaking Step
Testing Using 10 ml Test Sample Solution 1 With 4 Hour Soaking Time

| NR/NR | Lens | | |
|---|---|---|---|
| Regimen | Sample | Microorganism | CFU |
| Without | A | Pseudomas aeruginosa ATCC 9027 | 0, 0, 0 |
| Shaking | | Staphylococcus aureus ATCC 6538 | 0, 1, 0 |
| | | Serratia marcescens ATCC 13880 | 0, 0, 0 |
| | | Fusarium solani ATCC 36031 | 8, 5, 6 |
| With 10 | A | Pseudomas aeruginosa ATCC 9027 | 0, 0, 0 |
| Second | | Staphylococcus aureus ATCC 6538 | 0, 0, 0 |
| Shake | | Serratia marcescens ATCC 13880 | 0, 0, 1 |
| Before Soak | | Fusarium solani ATCC 36031 | 7, 5, 1 |
| Without | B | Pseudomas aeruginosa ATCC 9027 | 0, 0, 1 |
| Shaking | | Staphylococcus aureus ATCC 6538 | 0, 0, 1 |
| | | Serratia marcescens ATCC 13880 | 0, 0, 22 |
| | | Fusarium solani ATCC 36031 | 1, 4, 0 |
| With 10 | B | Pseudomas aeruginosa ATCC 9027 | 0, 0, 0 |
| Second | | Staphylococcus aureus ATCC 6538 | 0, 0, 2 |
| Shake | | Serratia marcescens ATCC 13880 | 0, 0, 0 |
| Before Soak | | Fusarium solani ATCC 36031 | 1, 0, 1 |

<10 CFU = test passage
>10 CFU = test failure
CFU = colony forming units

### EXAMPLE 6

### “No Rub-No Rinse” Regimen Testing of Sample Solutions

A four-hour no rub and no rinse (NR/NR) regimen using 8 ml of Sample Solution 4 with a 5 second shaking step (ss) was conducted on Focus™ Monthly (CIBA Vision, Basel, Switzerland) Group IV lenses. The lenses were tested using Candida albicans ATCC10231. The test results for the regimen are set forth below in Table 6.

TABLE 6
Results of No Rub-No Rinse Regimen With A Shaking Step Using
8 ml Sample Solution With 4 Hour Soaking Time

| NR/NR Regimen | CFU |
|---|---|
| Solution 4 | 0, 4, 0 |

<10 CFU = test passage
>10 CFU = test failure
CFU = colony forming unit

### EXAMPLE 7

### “No Rub-No Rinse” Regimen Testing of a Commercial Solution

A four-hour no rub and no rinse (NR/NR) regimen using 10 ml of Complete Moisture Plus™ No Rub Solution (AMO, Irvine, Calif.) Lot E2970, Expiration May 2005, with a 10 second shaking step (ss) was conducted on Focus™ Monthly (CIBA Vision, Basel, Switzerland) Group IV lenses (Lens Sample A), Surevue™ (Johnson & Johnson, New Brunswick, N.J.) Group IV lenses (Lens Sample B) and PureVision™ (Bausch & Lomb Inc., Rochester, N.Y.) Group III lenses (Lens Sample C). The lenses were tested using Candida albicans ATCC 10231. The test results for the regimens are set forth below in Table 7.

TABLE 7
Results of No Rub-No Rinse Regimen With A Shaking Step Using
10 ml Commercial Solution With 4 Hour Soaking Time

| NR/NR Regimen | Lens Sample | CFU |
|---|---|---|
| With 10 Second Shake | A | TNTC, TNTC, TNTC |
| With 10 Second Shake | B | TNTC, TNTC, TNTC |
| With 10 Second Shake | C | TNTC, TNTC, TNTC |

TNTC = Too numerous to count
<10 CFU = test passage
>10 CFU = test failure
CFU = colony forming unit

### EXAMPLE 8

### “No Rub-No Rinse” Regimen Testing of a Commercial Solution

A six-hour no rub and no rinse (NR/NR) regimen using 10 ml of Opti-Free Express™ (Alcon Laboratories Inc., Fort Worth, Tex.), Lots 44102F, 36311F, 34864F, and 35526F, both with a 10 second shaking step (ss) and without a shaking step was conducted on Focus™ Monthly (CIBA Vision, Basel, Switzerland) Group IV lenses (Lens Sample A) and Surevue™ (Johnson & Johnson, New Brunswick, N.J.) Group IV lenses (Lens Sample B). The lenses were tested using Candida albicans ATCC10231. The test results for the regimens are set forth below in Table 8.

TABLE 8
Results of No Rub-No Rinse Regimen With And Without A Shaking Step
Using 10 ml Commercial Solution With 6 Hour Soaking Time

| | Lens | | |
|---|---|---|---|
| NR/NR Regimen | Sample | Lot No. | CFU |
| With 10 Second Shake | A | 44102F | >100, >100, >100 |
| With 10 Second Shake | B | | >100, >100, >100 |
| Without Shake | A | | >100, >100, >100 |
| Without Shake | B | | >100, >100, >100 |
| With 10 Second Shake | A | 36311F | >100, >100, >100 |
| With 10 Second Shake | B | | 37, 19, 34 |
| Without Shake | A | | >100, >100, >100 |
| Without Shake | B | | 58, 58, >100 |
| With 10 Second Shake | A | 34864F | >100, >100, >100 |
| With 10 Second Shake | B | | >100, >100, >100 |
| Without Shake | A | | >100, >100, >100 |
| Without Shake | B | | >100, >100, >100 |
| With 10 Second Shake | A | 35526F | >100, >100, >100 |
| With 10 Second Shake | B | | 23, 22, >100 |
| Without Shake | A | | >100, >100, >100 |
| Without Shake | B | | >100, >100, >100 |

TNTC = Too numerous to count
<10 CFU = test passage
>10 CFU = test failure
CFU = colony forming units

### EXAMPLE 9

### “No Rub-No Rinse” Regimen Testing of a Commercial Solution

A four-hour no rub and no rinse (NR/NR) regimen using 10 ml of Solocare Plus™ (Ciba Vision, Atlanta, Ga.), Lot 22561, with a 10 second shaking step (ss) was conducted on Focus™ Monthly (CIBA Vision, Basel, Switzerland) Group IV lenses (Lens Sample A) and Surevue™ (Johnson & Johnson, New Brunswick, N.J.) Group IV lenses (Lens Sample B). The lenses were tested using Candida albicans ATCC10231. The test results for the regimens are set forth below in Table 9.

TABLE 9
Results of No Rub-No Rinse Regimen With A Shaking Step Using
10 ml Commercial Solution With 4 Hour Soaking Time

| NR/NR Regimen | Lens Sample | CFU |
|---|---|---|
| With 10 Second Shake | A | >100, >100, >100 |
| With 10 Second Shake | B | >100, >100, >100 |

TNTC = Too numerous to count
<10 CFU = test passage
>10 CFU = test failure
CFU = colony forming units

### EXAMPLE 10

### “No Rub-No Rinse” Regimen Testing of a Commercial Solution

A four-hour no rub and no rinse (NR/NR) regimen using 10 ml of Complete Comfort Plus™ (AMO, Irvine, Calif.), Lot 17576, with a 10 second shaking step (ss) was conducted on Focus™ Monthly (CIBA Vision, Basel, Switzerland) Group IV lenses (Lens Sample A) and Surevue™ (Johnson & Johnson, New Brunswick, N.J.) Group IV lenses (Lens Sample B). The lenses were tested using Candida albicans ATCC10231. The test results for the regimens are set forth below in Table 10.

TABLE 10
Results of No Rub-No Rinse Regimen With A Shaking Step
Using 10 ml Commercial Solution With 4 Hour Soaking Time

| NR/NR Regimen | Lens Sample | CFU |
|---|---|---|
| With 10 Second Shake | A | >100, >100, >100 |
| With 10 Second Shake | B | >100, >100, >100 |

TNTC = Too numerous to count
<10 CFU = test passage
>10 CFU = test failure
CFU = colony forming units

### EXAMPLE 11

### Stand-Alone Biocidal Testing and “No Rub-No Rinse” Regimen with a Shaking Step Testing of Commercial Solution with Four FDA/ISO Challenge Microorganisms with Group IV Lenses

A four-hour no rub and no rinse (NR/NR) regimen using 10 ml of Schalcon Universal PLUS™ (Schalcon, Rome, Italy), lot 0320, with a 10 second shaking step (ss) was conducted on Surevue™ (Johnson & Johnson, New Brunswick, N.J.) Group IV lenses and tested against Pseudomas aeruginosa ATCC 9027, Staphylococcus aureus ATCC 6538, Serratia marcescens ATCC 13880, Candida albicans ATCC 10231 and Fusarium solani ATCC 36031. The test results for the regimens are set forth below in Table 11.

A Stand-Alone Biocidal study using 10 percent organic soil was also conducted whereby the samples were tested against Pseudomas aeruginosa ATCC 9027, Staphylococcus aureus ATCC 6538, Candida albicans ATCC 10231 and Fusarium solani ATCC 36031. The results of the Stand-Alone Biocidal study are also set forth below in Table 11.

TABLE 11
Efficacy of Various Test Solutions in No Rub/No Rinse Regimen
with a Shaking Step and Stand-Alone Biocidal Testing

| TEST | CFU |
|---|---|
| NR/NR Regimen | |
| 4 Hr soak/10 ml/10 ss | |
| Pseudomas aeruginosa ATCC 9027 | >100, >100, >100 |
| Staphylococcus aureus ATCC 6538 | >100, >100, >100 |
| Serratia marcescens ATCC13880 | >100, >100, >100 |
| Candida albicans ATCC 10231 | >100, >100, >100 |
| Fusarium solani ATCC 36031 | >100, >100, >100 |

<10 CFU = test passage
>10 CFU = test failure
CFU = colony forming units

| Stand-Alone Biocidal | |
|---|---|
| (10% organic soil) | Log Reduction |
| Pseudomas aeruginosa | |
| 1 Hour Soaking Time | 1.3 |
| 4 Hour Soaking Time | 1.6 |
| Staphylococcus aureus | |
| 1 Hour Soaking Time | 2.2 |
| 4 Hour Soaking Time | 2.4 |
| Candida albicans | |
| 1 Hour Soaking Time | 0.1 |
| 4 Hour Soaking Time | 0.3 |
| Fusarium solani | |
| 1 Hour Soaking Time | 1.0 |
| 4 Hour Soaking Time | 0.1 |

Log Reduction: >= 100 percent kill

### EXAMPLE 12

### Stand-Alone Biocidal Testing and “No Rub-No Rinse” Regimen with a Shaking Step or Revolving Step Testing of Commercial Solution with Five of FDA/ISO Challenge Microorganisms with Group I, III and IV Lenses

A four-hour no rub and no rinse (NR/NR) regimen using 10 ml of Cyclean™ (Sauflon, Twickenhan, England), lot 54560, expiration April 2005, with a 10 second shaking step (ss) or 10 second revolving lens case step (rs) was conducted on Focus™ Night and Day (CIBA Vision, Basel, Switzerland) Group I lenses, PureVision™ (Bausch & Lomb Inc., Rochester, N.Y.) Group III lenses, Focus™ Monthly (CIBA Vision, Basel, Switzerland) Group IV lenses, and Surevue™ (Johnson & Johnson, New Brunswick, N.J.) Group IV lenses, and tested against Candida albicans ATCC 10231. The test results for the regimens are set forth below in Table 12. A Stand-Alone Biocidal study using 10 percent organic soil was also conducted whereby the samples were tested against Pseudomas aeruginosa ATCC 9027, Staphylococcus aureus ATCC 6538, Serratia marcescens ATCC13880, Candida albicans ATCC 10231 and Fusarium solani ATCC 36031. The results of the Stand-Alone Biocidal study are also set forth below in Table 12.

TABLE 12
Efficacy of Various Test Solutions in No Rub/No Rinse Regimen with
a Revolving Lens Case vs. a Shaking Step and Stand-Alone Biocidal
Testing

| TEST | CFU |
|---|---|
| NR/NR Regimen with Focus Monthly | TNTC |
| 4 Hr soak/10 ml/10 sr | |
| NR/NR Regimen with PureVision | TNTC |
| 4 Hr soak/10 ml/10 sr | |
| NR/NR Regimen with Focus Monthly | TNTC |
| 4 Hr soak/10 ml/10 ss | |
| NR/NR Regimen with Surevue | TNTC |
| 4 Hr soak/10 ml/10 ss | |
| NR/NR Regimen with PureVision | TNTC |
| 4 Hr soak/10 ml/10 ss | |
| NR/NR Regimen with Focus Night & Day | TNTC |
| 4 Hr soak/10 ml/10 ss | |

ss = seconds of shaking
sr = seconds of revolving
<10 CFU = test passage
>10 CFU = test failure
CFU = colony forming units
TNTC = too numerous to count

| Stand-Alone Biocidal with 4 Hour Soak | |
|---|---|
| (10% organic soil) | Log Reduction |
| Pseudomas aeruginosa | |
| 1 Hour Soaking Time | 4.8 |
| 4 Hour Soaking Time | >4.8 |
| Staphylococcus aureus | |
| 1 Hour Soaking Time | 4.7 |
| 4 Hour Soaking Time | >4.7 |
| Serratia marcescens | |
| 1 Hour Soaking Time | 4.4 |
| 4 Hour Soaking Time | 4.7 |
| Candida albicans | |
| 1 Hour Soaking Time | 0.3 |
| 4 Hour Soaking Time | 0.5 |
| Fusarium solani | |
| 1 Hour Soaking Time | 0.6 |
| 4 Hour Soaking Time | 0.6 |

Log Reduction: >= 100 percent kill

While there is shown and described herein compositions for lens care solutions, and methods of making and using the same in a no rub and no rinse regimen, it will be manifest to those skilled in the art that various modifications may be made without departing from the spirit and scope of the underlying inventive concept. The present invention is likewise not intended to be limited to particular ophthalmic solutions or methods described herein except insofar as indicated by the scope of the appended claims.

1. A composition comprising:
a volume of one or more polyols effective to achieve a composition osmolarity of 220 to 380 mOsm/kg;
one or more hydroxyalkylamines;
one or more polymeric surfactants having a HLB of 20 or greater; and
one or more disinfecting agents effective to achieve a no-rub and no-rinse regimen for contact lens disinfection.

2. The composition of claim 1 wherein said one or more polyols includes glycerin present in an amount of approximately 0.5 weight percent or greater.

3. The composition of claim 1 wherein said one or more hydroxyalkylamines are present in a total concentration of about 0.5 to 2.0 weight percent.

4. The composition of claim 1 wherein said one or more hydroxyalkylamines are present in a total concentration of about 1.0 weight percent.

5. The composition of claim 1 wherein said one or more polymeric surfactants include Pluronic or Tetronic.

6. The composition of claim 1 wherein said one or more polymeric surfactants include Pluronic F38 and Tetronic 908.

7. The composition of claim 1 wherein said one or more disinfecting agents include PHMB or Alexidine.

8. The composition of claim 1 wherein said one or more disinfecting agents are present in a total concentration of about 3 ppm to 6 ppm.

9. The composition of claim 1 wherein said one or more disinfecting agents are present in a concentration of about 0.5 ppm PHMB and about 3.0 ppm Alexidine.

10. The composition of claim 1 wherein said one or more disinfecting agents are present in a concentration of about 0.7 ppm PHMB and about 4.0 ppm Alexidine.

11. The composition of claim 1 wherein said one or more disinfecting agents is PHMB in a concentration of about 0.5 ppm to about 1.1 ppm.

12. The composition of claim 1 wherein said one or more disinfecting agents is Alexidine in a concentration of about 4.0 ppm to about 6.0 ppm.

13. The composition of claim 1 wherein said composition has a pH within a range of about 6.0 to 8.0.

14. A no-rub and no-rinse contact lens cleaning and disinfecting solution comprising:
a volume of one or more polyols effective to achieve a composition osmolarity of 220 to 380 mOsm/kg;
one or more hydroxyalkylamines;
one or more polymeric surfactants having a HLB of 20 or greater; and
one or more disinfecting agents effective to achieve a no-rub and no-rinse regimen for contact lens disinfection.

15. The solution of claim 14 wherein said one or more polyols includes glycerin present in an amount of approximately 0.5 weight percent or greater.

16. The solution of claim 14 wherein said one or more hydroxyalkylamines are present in a total concentration of about 0.5 to 2.0 weight percent.

17. The solution of claim 14 wherein said one or more hydroxyalkylamines are present in a total concentration of about 1.0 weight percent.

18. The solution of claim 14 wherein said one or more polymeric surfactants include Pluronic or Tetronic.

19. The solution of claim 14 wherein said one or more polymeric surfactants include Pluronic F38 and Tetronic 908.

20. The solution of claim 14 wherein said one or more disinfecting agents include PHMB or Alexidine.

21. The solution of claim 14 wherein said one or more disinfecting agents are present in a total concentration of about 3 ppm to 6 ppm.

22. The solution of claim 14 wherein said one or more disinfecting agents are present in a concentration of about 0.5 ppm PHMB and about 3.0 ppm Alexidine.

23. The solution of claim 14 wherein said one or more disinfecting agents are present in a concentration of about 0.7 ppm PHMB and about 4.0 ppm Alexidine.

24. The solution of claim 14 wherein said one or more disinfecting agents is PHMB in a concentration of about 0.5 ppm to about 1.1 ppm.

25. The solution of claim 14 wherein said one or more disinfecting agents is Alexidine in a concentration of about 4.0 ppm to about 6.0 ppm.

26. The solution of claim 14 wherein said composition has a pH within a range of about 6.0 to 8.0.

27. A method of making the composition of claim 1 comprising:
combining a volume of one or more polyols effective to achieve a composition osmolarity of 220 to 380 mOsm/kg;
one or more hydroxyalkylamines;
one or more polymeric surfactants having a HLB of 20 or greater; and
one or more disinfecting agents effective to achieve a no-rub and no-rinse regimen for contact lens disinfection.

28. A method of making the solution of claim 2 comprising:
combining a volume of one or more polyols effective to achieve a composition osmolarity of 220 to 380 mOsm/kg;
one or more hydroxyalkylamines;
one or more polymeric surfactants having a HLB of 20 or greater; and
one or more disinfecting agents effective to achieve a no-rub and no-rinse regimen for contact lens disinfection.

29. The method of claim 27 or 28 wherein said one or more polyols includes glycerin is present in an amount of approximately 0.5 weight percent or greater.

30. The method of claim 27 or 28 wherein said one or more hydroxyalkylamines are present in a total concentration of about 0.5 to 2.0 weight percent.

31. The method of claim 27 or 28 wherein said one or more hydroxyalkylamines are present in a total concentration of about 1.0 weight percent.

32. The method of claim 27 or 28 wherein said one or more polymeric surfactants include Pluronic or Tetronic.

33. The method of claim 27 or 28 wherein said one or more polymeric surfactants include Pluronic F38 and Tetronic 908.

34. The method of claim 27 or 28 wherein said one or more disinfecting agents include PHMB or Alexidine.

35. The method of claim 27 or 28 wherein said one or more disinfecting agents are present in a total concentration of about 3 ppm to 6 ppm.

36. The method of claim 27 or 28 wherein said one or more disinfecting agents are present in a concentration of about 0.5 ppm PHMB and about 3.0 ppm Alexidine.

37. The method of claim 27 or 28 wherein said one or more disinfecting agents are present in a concentration of about 0.7 ppm PHMB and about 4.0 ppm Alexidine.

38. The method of claim 27 or 28 wherein said one or more disinfecting agents is PHMB in a concentration of about 0.5 ppm to about 1.1 ppm.

39. The method of claim 27 or 28 wherein said one or more disinfecting agents is Alexidine in a concentration of about 4.0 ppm to about 6.0 ppm.

40. The method of claim 27 or 28 wherein said composition has a pH within a range of about 6.0 to 8.0.

41. A method of using the composition of claim 1 in a no-rub and no-rinse regimen comprising:
adding a solution of said composition to a case containing a contact lens prior to shaking or revolving said case containing said solution and said contact lens.

42. A method of using the solution of claim 2 in a no-rub and no-rinse regimen comprising:
adding said solution to a case containing a contact lens prior to shaking or revolving said case containing said solution and said contact lens.

43. A method of using the composition of claim 1 comprising:
shaking or revolving a contact lens in said composition prior to soaking said contact lens in said composition for a period of time sufficient to disinfect said contact lens.

44. A method of using the solution of claim 2 comprising:
shaking or revolving a contact lens in said solution prior to soaking said contact lens in said solution for a period of time sufficient to disinfect said contact lens.

45. The method of claim 41, 42, 43 or 44 wherein said one or more polyols includes glycerin present in an amount of approximately 0.5 weight percent or greater.

46. The method of claim 41, 42, 43 or 44 wherein said one or more hydroxyalkylamines are present in a total concentration of about 0.5 to 2.0 weight percent.

47. The method of claim 41, 42, 43 or 44 wherein said one or more hydroxyalkylamines are present in a total concentration of about 1.0 weight percent.

48. The method of claim 41, 42, 43 or 44 wherein said one or more polymeric surfactants include Pluronic or Tetronic.

49. The method of claim 41, 42, 43 or 44 wherein said one or more polymeric surfactants include Pluronic F38 and Tetronic 908.

50. The method of claim 41, 42, 43 or 44 wherein said one or more disinfecting agents include PHMB or Alexidine.

51. The method of claim 41, 42, 43 or 44 wherein said one or more disinfecting agents are present in a total concentration of about 3 ppm to 6 ppm.

52. The method of claim 41, 42, 43 or 44 wherein said one or more disinfecting agents are present in a concentration of about 0.5 ppm PHMB and about 3.0 ppm Alexidine.

53. The method of claim 41, 42, 43 or 44 wherein said one or more disinfecting agents are present in a concentration of about 0.7 ppm PHMB and about 4.0 ppm Alexidine.

54. The method of claim 41, 42, 43 or 44 wherein said one or more disinfecting agents is PHMB in a concentration of about 0.5 ppm to about 1.1 ppm.

55. The method of claim 41, 42, 43 or 44 wherein said one or more disinfecting agents is Alexidine in a concentration of about 4.0 ppm to about 6.0 ppm.

56. The method of claim 41, 42, 43 or 44 wherein said composition has a pH within a range of about 6.0 to 8.0.
